SEQUENCE LISTING (2 sequences)

SEQ ID NO: 1 - DNA, 1201 bp, Homo sapien
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 2 - protein, 212 aa, Homo sapien
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

(19) United States
(12) Patent Application Publication
(10) Pub. No.: US 2011/0262387 A1
(43) Pub. Date: Oct. 27, 2011

(54) METHODS AND MATERIALS FOR REDUCING OR SUPPRESSING AMYLOID DEPOSITION

(76) Inventors: Pritam Das, Ponte Vedra Beach, FL (US); Todd E. Golde, Newberry, FL (US); Paramita Chakrabarty, Gainesville, FL (US)

(21) Appl. No.: 13/016,607

(22) Filed: Jan. 28, 2011

Related U.S. Application Data
(60) Provisional application No. 61/299,061, filed on Jan. 28, 2010.

(51) Int. Cl. A61K 38/20; A61P 25/28; A61K 48/00

(52) U.S. Cl. 424/85.2; 514/44 R

(57) ABSTRACT

This document relates to methods and materials for treating diseases and disorders that are caused by or associated with amyloid or amyloid-like proteins, such as Alzheimer's disease. For example, methods and materials for using interleukin-6 to reduce or suppress the deposition of Aβ are provided.

### CROSS-REFERENCE TO RELATED APPLICATIONS

This application claims the benefit of priority to U.S. Application Ser. No. 61/299,061, filed Jan. 28, 2010. The disclosure of the prior application is considered part of (and is incorporated by reference in) the disclosure of this application.

### BACKGROUND

1. Technical Field

This document relates to methods and materials for reducing or suppressing amyloid deposition. For example, this document provides methods and materials for using interleukin-6 (IL-6) polypeptides and/or IL-6 analogs to treat diseases or disorders that are caused by or associated with amyloid or amyloid-like proteins (e.g., Alzheimer's disease).

2. Background Information

Amyloidosis is not a single disease entity but rather a diverse group of progressive disease processes characterized by extracellular tissue deposits of a waxy, starch-like protein called amyloid, which accumulates in one or more organs or body systems. As the amyloid deposits build up, they begin to interfere with the normal function of the organ or body system. There are at least 15 different types of amyloidosis. Some of these diseases include Alzheimer's disease (AD), mild cognitive impairment (MCI), Lewy body dementia (LBD), amyotrophic lateral sclerosis (ALS), inclusion-body myositis (IBM), and macular degeneration (e.g., age-related macular degeneration (AMD)). Although the pathogenesis of these diseases may be diverse, their characteristic deposits contain many shared molecular constituents. To a significant degree, this may be attributable to the local activation of pro-inflammatory pathways thereby associated with the concurrent deposition of activated complement components, acute phase reactants, immune modulators, and other inflammatory mediators (McGreer et al., Tohoku J. Exp. Med., 174:269 (1994)).

AD is the most common form of age-related neurodegenerative illness. The defining pathological hallmarks of AD are the presence of neurofibrillary tangles and senile plaques in the brain. Amyloid β polypeptides (Aβ) are the major constituents of amyloid plaques and are derived from altered processing of amyloid precursor proteins (APP). Scientific evidence demonstrates that an increase in the production and accumulation of beta-amyloid protein in plaques leads to nerve cell death, which contributes to the development and progression of AD.

Another pathologic feature of AD is reactive gliosis, a phenomenon that is especially prominent in the vicinity of extracellular Aβ plaques and is characterized by astrocyte activation (Wyss-Coray, T. Nat. Med., 12:1005 (2006)). Amyloid deposition has also been suggested to lead to neuroinflammation, creating a positive feedback loop resulting in further amyloid accumulation and chronic inflammation, possibly mediated by a proinflammatory stimuli.

### SUMMARY

This document provides methods and materials related to reducing or suppressing amyloid deposition. For example, this document provides methods and materials related to the use of IL-6 polypeptide or IL-6 analogs to reduce or suppress protein deposition (e.g., amyloid deposition) in mammals (e.g., humans) with an amyloid disease of the CNS. As described herein, IL-6 polypeptides can be used to treat amyloidosis. For example, a method provided herein for reducing amyloid deposition can include administering a therapeutically effective amount of an IL-6 polypeptide (e.g., a human IL-6 polypeptide) or an analog thereof to a mammal. In some cases, the IL-6 polypeptide can be a recombinant human IL-6 (rIL-6) polypeptide. In some cases, an analog of an IL-6 polypeptide derived from rIL-6, or a synthetic polypeptide analog of IL-6, having the amyloid deposition reducing activity of IL-6 can be used.

The therapeutically effective amount of IL-6 or IL-6 analog can be administered enterally or parenterally including intrathecally. In some cases, IL-6 or analog thereof can be administered subcutaneously.

In some cases, IL-6 or IL-6 analogs can be administered in combination with an agent known to increase the permeability of the blood brain barrier. In some cases, IL-6 or an IL-6 analog can be modified to enhance blood brain barrier permeability. For example, an IL-6 polypeptide can be modified to include a polyamine modification. Other methods that can be used to increase blood brain barrier permeability include, without limitation, those described elsewhere. See, e.g., U.S. Pat. Nos. 5,260,308 and 7,279,149.

In another aspect, this document features a method for treating AD. Such a method can include administering a therapeutically effective amount of an IL-6 polypeptide or an analog thereof to a patient who has been diagnosed with, or who is suspected of acquiring, AD. In some cases, the treatment can include using recombinant techniques to produce a vector molecule containing a nucleic acid encoding an IL-6 polypeptide (e.g., a human IL-6 polypeptide) and infecting a target cell of the mammalian host with the vector molecule containing the nucleic acid. The vector molecule can be any molecule capable of being delivered and maintained within the target cell or tissue such that the nucleic acid encoding IL-6 is expressed (e.g., stably expressed). The vector molecule can be a viral or retroviral vector molecule (e.g., a recombinant adeno-associated virus serotype 1 (AAV1)) or a non-viral, plasmid DNA molecule.

As described herein, an AAV1 vector containing an IL-6 coding sequence can be ligated downstream of the cytomegalovirus (CMV) promoter and used to treat AD as demonstrated herein via injection into the brains of mice that are a transgenic model of AD. The results provided herein demonstrate that IL-6 can enhance the phenotypic and immunological activation of glia and that IL-6 does not promote Aβ accumulation but rather limits Aβ deposition.

In another aspect, this document features an article of manufacture having packaging material and a pharmaceutical agent contained within the packaging material. The pharmaceutical agent can be therapeutically effective for reducing or suppressing amyloid deposition in central nervous system (CNS) tissue. The packaging material can contain a label indicating that the pharmaceutical agent can be used to reduce or suppress amyloid deposition in the CNS tissue. The pharmaceutical agent can be IL-6 or an analog thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Levels of mIL-6 in injected mouse brains at ages P0 (a), P2 (b), or 4 months (c) analyzed by sandwich ELISA using RIPA-soluble brain lysates.

FIG. 2. GFAP immunostaining revealed astrogliosis in brains of TgCRND8 mice that were injected with either AAV1-mIL-6 or AAV1-EGFP at ages P0 (a), P2 (b), or 4 months (c).

FIG. 3. AAV1-mIL-6 expression in transgenic CRND8 mice resulted in extensive induction of astrogliosis. Up-regulation of activated astrocytes in the cortex of P0→mIL-6-injected TgCRND8 mice (a) as compared with control TgCRND8 mice and (b) as detected by GFAP immunofluorescent staining of free-floating fixed sections. Scale bars=50 μm.

FIG. 4. AAV1-mIL-6 expression in transgenic CRND8 mice resulted in extensive induction of microgliosis. Up-regulation of activated microglia in the cortex of P0→mIL-6-injected TgCRND8 mice (a) as compared with control TgCRND8 mice and (b) as detected by Iba-1 immunofluorscent staining of free-floating fixed sections. Scale bars=50 μm.

FIG. 5. Quantification of Aβ plaque immunoreactivity in brain sections stained with pan-Aβ1-16 antibody (mAb 33.1.1) in the hippocampus of P0→5 mo IL-6-expressing, p2→5 mo IL-6-expressing, and age-matched P0→5 mo EGFP-expressing TgCRND8 mice. Aβ deposition in AAV1-mIL-6-expressing TgCRND8 mice is attenuated. There was a significant decrease in total forebrain Aβ as well as hippocampal Aβ plaque burdens in both P0→5 mo and P2→5 mo injection groups compared with control mice.

FIG. 6. mIL-6 treated primary microglia were more efficient in the uptake of fAβ42-Hilyte488 (green fluorescence) as compared with unstimulated glia. Blue fluorescence indicates DAPI-stained glial nuclei.

FIG. 7. Microglia cells with internalized Aβ42-Hilyte488 were quantified by FACS. Distribution of positive cells in mIL-6 stimulated microglial cells as compared with control unstimulated cells.

FIG. 8 contains a listing of a nucleic acid sequence (SEQ ID NO:1) that encodes a human IL-6 polypeptide.

FIG. 9 contains a listing of an amino acid sequence (SEQ ID NO:2) of a human IL-6 polypeptide.

### DETAILED DESCRIPTION

This document provides methods and materials related to reducing or suppressing protein deposition in mammals (e.g., humans) with an amyloid disease of the CNS. For example, this document provides methods and materials for using IL-6 polypeptides to treat AD. The methods and materials provided herein can include administering a therapeutically effective amount of IL-6 or an analog thereof to a mammal. Any type of mammal can be treated using the methods and materials provided herein. For example, an IL-6 polypeptide or IL-6 analog can be administered to a human, dog, cat, horse, cow, pig, mouse, or rat under conditions wherein the level of amyloid deposition within CNS tissue of the mammal is reduce and/or under conditions wherein the accumulation of amyloid deposition within CNS tissue of a mammal is suppressed.

IL-6 is a cytokine of approximately 26 kD that is synthesized by mononuclear phagocytes, vascular endothelial cells, fibroblasts, and other cells in response to IL-1 and, to a lesser extent, TNF. IL-6 is one of the major mediators of the immune response, with pleiotropic effects on many different target cells. IL-6 activates microglia, immune cells of the brain. Molecules that take part in the inflammatory cascade are of great interest, because inflammation has repeatedly been suggested to be associated with the neurodegenerative process characteristic of the AD brain (Fassbender et al., Neurobiol. Aging, 21:433 (2000)). Astrocyte-produced IL-6 has been proposed to play a role in augmenting intracerebral immune responses. Increased levels of IL-6 have been reported in the plasma, cerebrospinal fluid, and brain parenchyma in patients with AD (Licastro et al., J. Neuroimmunol., 103:97 (2000); Galimberti et al., J. Neurol., 255:539 (2008); and Baranowska-Bik et al., Neuro. Endocrinol. Lett., 29:75 (2008)). Over-expression of cytokines and other inflammatory molecules are common features of AD brain pathology (McGeer and McGeer, Neurobiol. Aging, 22:799 (2001)).

As described herein, in vivo administration of AAV1 expressing IL-6 suppresses the deposition of Aβ. The transgenic mouse model utilized, Tg2576 mice expressing mutant human APP (KM670/671NL) gene under the control of hamster prion promoter, were injected with AAV1-mIL-6 or AAV1-EGFP. Experiments in primary mouse microglial cells, as described herein, demonstrate that when treated with recombinant mIL-6, the cells exhibit increased uptake of labeled fibrillar Aβ. This reduction in Aβ deposition is likely mediated by enhanced microglial clearance of the Aβ deposits.

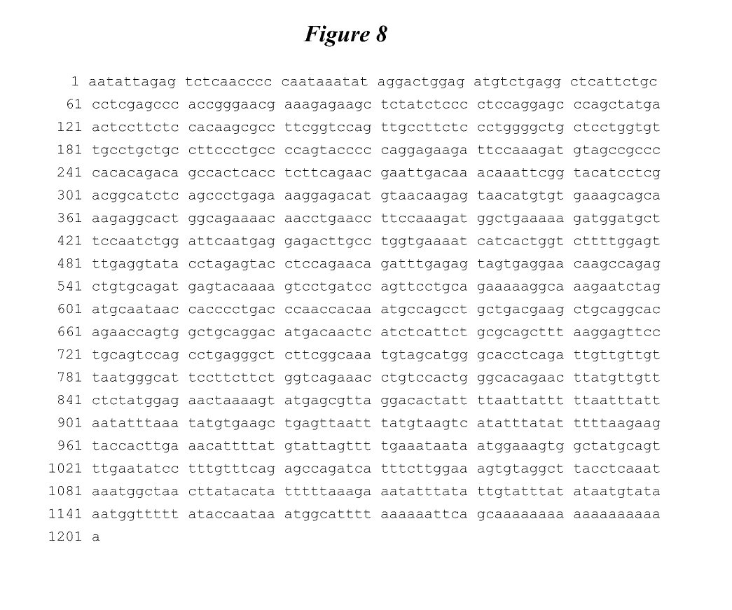

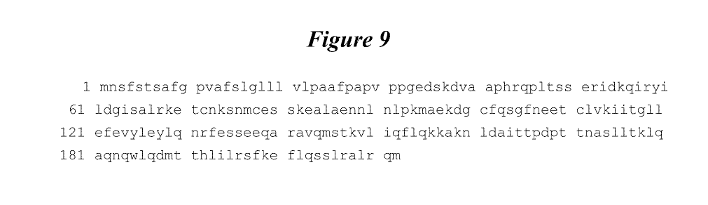

The methods and materials provided herein can be used to reduce amyloid deposition within CNS tissue of a mammal or to suppress accumulation of amyloid deposition within CNS tissue of a mammal. For example, an IL-6 polypeptide or an IL-6 analog can be administered to a mammal under conditions wherein the IL-6 polypeptide reduces amyloid deposition within the mammal's CNS tissue. Examples of IL-6 polypeptides include, without limitation, human IL-6 polypeptides, mouse IL-6 polypeptides, monkey IL-6 polypeptides, bovine IL-6 polypeptides, equine IL-6 polypeptides, and porcine IL-6 polypeptides. Additional examples of IL-6 polypeptides are described elsewhere (Fassbender et al., Neurobiol. Aging, 21:433 (2000); Licastro et al., J. Neuroimmunol., 103:97 (2000); Galimberti et al., J. Neurol., 255:539 (2008); Baranowska-Bik et al., Neuro. Endocrinol. Lett., 29:75 (2008); and McGeer and McGeer, Neurobiol. Aging, 22:799 (2001)). A nucleic acid sequence encoding a human IL-6 polypeptide is set forth in FIG. 8, and an amino acid sequence of a human IL-6 polypeptide is set forth in FIG. 9.

Examples of IL-6 analogs include, without limitation, fragments of mature IL-6 polypeptides that retain the ability to reduce amyloid deposition within CNS tissue of a mammal and/or to suppress accumulation of amyloid deposition within CNS tissue of a mammal, polypeptides having the amino acid sequence of an IL-6 polypeptide with one or more conservative amino acid changes provided that the polypeptide containing the one or more conservative amino acid changes retains the ability to reduce amyloid deposition within CNS tissue of a mammal and/or to suppress accumulation of amyloid deposition within CNS tissue of a mammal, and polypeptides having the amino acid sequence of an IL-6 polypeptide with one or more chemical modifications (e.g., a chemical modification such as PEGylation, acylation, and citrullination) provided that the polypeptide containing the one or more chemical modifications retains the ability to reduce amyloid deposition within CNS tissue of a mammal and/or to suppress accumulation of amyloid deposition within CNS tissue of a mammal. In some cases, an IL-6 analog can be a polypeptide that, compared to a mature IL-6 polypeptide, is a fragment with or without conservative amino acid changes and/or with or without chemical modifications. For example, an IL-6 analog can be a PEGylated fragment of a mature human IL-6 polypeptides provided that the PEGylated fragment retains the ability to reduce amyloid deposition within CNS tissue of a mammal and/or to suppress accumulation of amyloid deposition within CNS tissue of a mammal. Additional examples of polypeptides that can be used as IL-6 analogs as described herein include, without limitation, those polypeptides described elsewhere (Yawata et al., EMBO J., 12(4):1705-12 (1993); Hammacher et al., Protein Sci., 3(12):2280-93 (1994); and Brakenhoff et al., J. Immunol., 145(2):561-8 (1990)). Any appropriate method can be used to confirm that a polypeptide designed to be an IL-6 analog has the ability to reduce amyloid deposition within CNS tissue of a mammal and/or to suppress accumulation of amyloid deposition within CNS tissue of a mammal. For example, the ability of IL-6 polypeptide fragments of various sizes from various regions of the IL-6 molecule can be assessed for the ability to reduce amyloid deposition within CNS tissue of a mammal and/or to suppress accumulation of amyloid deposition within CNS tissue of a mammal using the experimental models described herein.

In some cases, prior to administering an IL-6 polypeptide or an IL-6 analog to a mammal (e.g., a human), the mammal can be identified as being in need of treatment with an IL-6 polypeptide or an IL-6 analog. Any appropriate method can be used to identify a mammal as being in need of treatment with an IL-6 polypeptide or an IL-6 analog. For example, standard clinical diagnostic tests used to diagnose mammals as having an amyloidosis condition can be used to identify the mammal as being in need of treatment with an IL-6 polypeptide or an IL-6 analog. In some cases, humans diagnosed or suspected as having an amyloidosis condition such as Alzheimer's disease can be identified as being in need of treatment with an IL-6 polypeptide or an IL-6 analog.

Once identified, the mammal can be administered an IL-6 polypeptide or an IL-6 analog. Any appropriate route of administration can be used to administer an IL-6 polypeptide or an IL-6 analog to a mammal. For example, a composition containing an IL-6 polypeptide or an IL-6 analog (or a combination thereof) and formulated in a manner appropriate for administration enterally or parenterally can be administered enterally or parenterally (e.g., intrathecally, intravenously, or intramuscularly). In some cases, a composition containing an IL-6 polypeptide or an IL-6 analog (or a combination thereof) can be administered in a manner that results in reduced amyloid deposition within CNS tissue of a mammal and/or suppressed accumulation of amyloid deposition within CNS tissue of a mammal. In some cases, a composition containing an IL-6 polypeptide or an IL-6 analog (or a combination thereof) can be administered in a manner that induces gliosis (e.g., massive reactive gliosis), induces neuroinflammation, and/or enhances Aβ phagocytosis by microglia within the mammal.

In some cases, an IL-6 polypeptide or an IL-6 analog can be administered to a mammal by administering a composition containing the IL-6 polypeptide or IL-6 analog. For example, a composition containing an IL-6 polypeptide or IL-6 analog can be directly injected into CNS tissue of a mammal to be treated. In some cases, a composition containing an IL-6 polypeptide or IL-6 analog can be directly administered to a region of the mammal outside the mammal's CNS tissue. In such cases, techniques such as those described in U.S. Pat. No. 5,260,308 and/or U.S. Pat. No. 7,279,149 can be used to aid in delivery of the IL-6 polypeptide or IL-6 analog to CNS tissue.

In some cases, an IL-6 polypeptide or an IL-6 analog can be administered to a mammal by administering a composition containing nucleic acid that encodes the IL-6 polypeptide or IL-6 analog. In such cases, the nucleic acid can be expressed by cells within the mammal such that the IL-6 polypeptide or IL-6 analog is produced. The nucleic acid encoding an IL-6 polypeptide or IL-6 analog can be configured to be a component of any appropriate vector designed to express the encoded IL-6 polypeptide or IL-6 analog. For example, nucleic acid encoding an IL-6 polypeptide or IL-6 analog can be configured into a viral vector or a non-viral vector.

Vectors for administering nucleic acids (e.g., a nucleic acid encoding an IL-6 polypeptide or IL-6 analog) to a mammal can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). Virus-based nucleic acid delivery vectors are typically derived from animal viruses, such as adenoviruses, adeno-associated viruses, retroviruses, lentiviruses, vaccinia viruses, herpes viruses, and papilloma viruses. Lentiviruses are a genus of retroviruses that can be used to infect cells. Adenoviruses contain a linear double-stranded DNA genome that can be engineered to inactivate the ability of the virus to replicate in the normal lytic life cycle. Adenoviruses and adeno-associated viruses can be used to infect cells within a mammal.

Vectors for nucleic acid delivery can be genetically modified such that the pathogenicity of the virus is altered or removed. The genome of a virus can be modified to increase infectivity and/or to accommodate packaging of a nucleic acid, such as a nucleic acid encoding an IL-6 polypeptide or IL-6 analog. A viral vector can be replication-competent or replication-defective, and can contain fewer viral genes than a corresponding wild-type virus.

In addition to nucleic acid encoding an IL-6 polypeptide or IL-6 analog, a viral vector can contain regulatory elements operably linked to a nucleic acid encoding an IL-6 polypeptide or IL-6 analog. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding an IL-6 polypeptide or IL-6 analog. A promoter can be constitutive or inducible (e.g., in the presence of tetracycline), and can affect the expression of a nucleic acid encoding an IL-6 polypeptide or IL-6 analog in a general or tissue-specific manner. Tissue-specific promoters include, without limitation, enolase promoters, prion protein (PrP) promoters, and tyrosine hydroxylase promoters.

As used herein, “operably linked” refers to positioning of a regulatory element in a vector relative to a nucleic acid in such a way as to permit or facilitate expression of the encoded polypeptide. For example, a viral vector can contain a neuronal-specific enolase promoter and a nucleic acid encoding an IL-6 polypeptide or IL-6 analog. In this case, the enolase promoter is operably linked to a nucleic acid encoding an IL-6 polypeptide or IL-6 analog such that it drives transcription in neuronal cells.

A nucleic acid encoding an IL-6 polypeptide or IL-6 analog also can be administered to cells within a mammal using non-viral vectors in a manner similar to those described elsewhere. See, for example, Gene Therapy Protocols (Methods in Molecular Medicine), edited by Jeffrey R. Morgan, Humana Press, Totowa, N.J. (2002). For example, a nucleic acid encoding an IL-6 polypeptide or IL-6 analog can be administered to a mammal by direct injection (e.g., an intrathecal injection) of nucleic acid molecules (e.g., plasmids) comprising nucleic acid encoding an IL-6 polypeptide or IL-6 analog, or by administering nucleic acid molecules complexed with lipids, polymers, or nanospheres.

A nucleic acid encoding an IL-6 polypeptide or IL-6 analog can be produced by standard techniques, including, without limitation, common molecular cloning techniques, polymerase chain reaction (PCR) techniques, chemical nucleic acid synthesis techniques, and combinations of such techniques. For example, PCR or RT-PCR can be used with oligonucleotide primers designed to amplify nucleic acid (e.g., genomic DNA or RNA) encoding an IL-6 polypeptide or fragment thereof.

A composition including an IL-6 polypeptide, IL-6 analog, or combination thereof or a composition including a nucleic acid encoding an IL-6 polypeptide, IL-6 analog, or combination thereof can be administered to a mammal in any amount, at any frequency, and for any duration effective to achieve a desired outcome (e.g., to reduce amyloid deposition within CNS tissue of a mammal and/or to suppress accumulation of amyloid deposition within CNS tissue of a mammal). An effective amount can vary, as recognized by those skilled in the art, depending on the severity of the condition, the route of administration, the age and general health condition of the subject, excipient usage, the possibility of co-usage with other therapeutic treatments such as use of other agents, and the judgment of the treating physician.

An effective amount of a composition containing an IL-6 polypeptide, IL-6 analog, or combination thereof (or nucleic acid encoding an IL-6 polypeptide, IL-6 analog, or combination thereof) can be any amount that reduces amyloid deposition within CNS tissue of a mammal and/or suppresses accumulation of amyloid deposition within CNS tissue of a mammal without producing significant toxicity to the mammal. If a particular mammal fails to respond to a particular amount, then the amount of IL-6 polypeptide, IL-6 analog, or combination thereof (or nucleic acid encoding an IL-6 polypeptide, IL-6 analog, or combination thereof) can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition may require an increase or decrease in the actual effective amount administered.

The frequency of administration can be any frequency that reduces amyloid deposition within CNS tissue of a mammal and/or suppresses accumulation of amyloid deposition within CNS tissue of a mammal without producing significant toxicity to the mammal. For example, the frequency of administration can be from about once a week to about three times a day, or from about twice a month to about six times a day, or from about twice a week to about once a day. The frequency of administration can remain constant or can be variable during the duration of treatment. A course of treatment with a composition containing an IL-6 polypeptide, IL-6 analog, or combination thereof (or nucleic acid encoding an IL-6 polypeptide, IL-6 analog, or combination thereof) can include rest periods. For example, a composition containing viruses having nucleic acid encoding an IL-6 polypeptide can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing an IL-6 polypeptide, IL-6 analog, or combination thereof (or nucleic acid encoding an IL-6 polypeptide, IL-6 analog, or combination thereof) can be any duration that reduces amyloid deposition within CNS tissue of a mammal and/or suppresses accumulation of amyloid deposition within CNS tissue of a mammal without producing significant toxicity to the mammal. Thus, the effective duration can vary from several days to several weeks, months, or years. In general, the effective duration for the treatment of AD can range in duration from several weeks to several years. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the condition.

In certain instances, a course of treatment and the severity of one or more symptoms related to an amyloidosis condition can be monitored. Any appropriate method can be used to determine whether or not the severity of a symptom of an amyloidosis condition is reduced. For example, brain imaging techniques and cognative tests can be used to assess one or more symptoms related to an amyloidosis condition at different time points.

The invention will be further described in the following examples, which do not limit the scope of the invention described in the claims.

### EXAMPLES

### Example 1

### AAV1-Mediated Expression of mIL-6 Leads to Widespread Gliosis in the Brains of TgCRND8 Mice, but Attenuates Aβ Deposition

Recombinant AAV1 preparation

rAAV1 viruses expression mIL-6 or EGFP under the control of the cytomegalovirus enhancer/chicken β-actin promoter, were generated by calcium-phosphate transfection of pAM/CBA-pI-WPRE-EGH, rAAV1 cis-plasmid pH21 (AAV1 helper plasmid), and pFA6 into a HEK293 cell line. At 48 h after transfection, cells were lysed in the presence of 0.5% sodium deoxycholate and 50 U/mL benzonase (Sigma) by repeated rounds of freeze/thaw at −80° C. and −20° C. The virus was isolated using a discontinuous Iodixanol gradient and then affinity purified on a HiTrap HQ column (GE Healthcare). Samples were eluted from the column and buffer exchanged to PBS using an Amicon Ultra 100 Centrifugation device (Millipore). The genomic titer of each virus was determined by quantitative PCR using the ABI7900 (Applied Biosystems). The viral DNA samples were prepared by treating the virus with DNase I (Invitrogen), heat inactivating the enzyme, and then digesting the protein coat with Proteinase K (Invitrogen), followed by a second heat inactivation. Samples were compared against a standard curve of supercoiled plasmid.

Mice and rAAV1 Injections

Tg2576 mice expressing mutant human APP (KM670/671NL) gene under the control of hamster prion promoter were injected with AAV1-mIL-6 or AAV1-EGFP. Cryoanesthetized mouse pups (0-12 h old (P0) or 36-48 h old (P2)) were bilaterally injected with 2 μL of AAV1 construct (10¹² particles/mL) in the cerebral ventricles. Negative control groups (total n=10) were noninjection (n=5) and AAV1-EGFP-injected (n=5) groups. Experimental groups consisted of mice injected with AAV1-mIL-6 on P0 or P2 (n=9-12 transgenics/group). Tg2576 injected or uninfected mice were euthanized after 3 mo (n=5 and 6, respectively). For stereotactic injections, mice (n=5-6/group) were anesthetized with 1.5% isoflurane in 1% oxygen and secured into a Kopf apparatus (Model 900 Small Animal Stereotactic Instrument, David Kopf Instruments, Tujunga, Calif., USA). The coordinates for injection were −1.7 caudal, −1.6 lateral, and −1.2 ventral from the bregma. A UMP2 Microsyringe Injector and Micro4 Controller (World Precision Instruments, Sarasota, Fla., USA) were used to inject 2 μL of virus at a constant rate over a 10 minute period. After an additional 10 min were allowed, the needle was raised slowly and the scalp incision was closed aseptically.

### Quantification of Aβ Deposition

Brain sections were immunohistochemically stained using pan-Aβ antibody 33.1.1. Stained sections were captured using the Scanscope XT image scanner (Aperio, Vista, Calif., USA) and analyzed using the ImageScope program. Plaque burden was calculated using the Positive Pixel Count program (Aperio). At least 3 sections/sample, 30 μm apart, were averaged by a blind observer to calculate plaque burden.

### Results

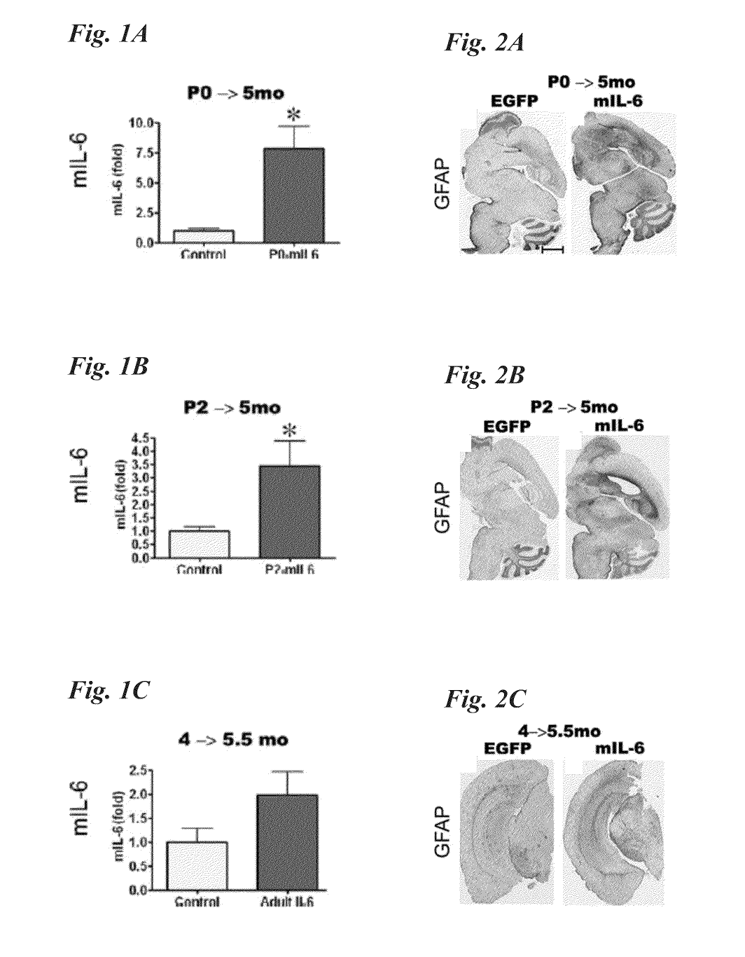

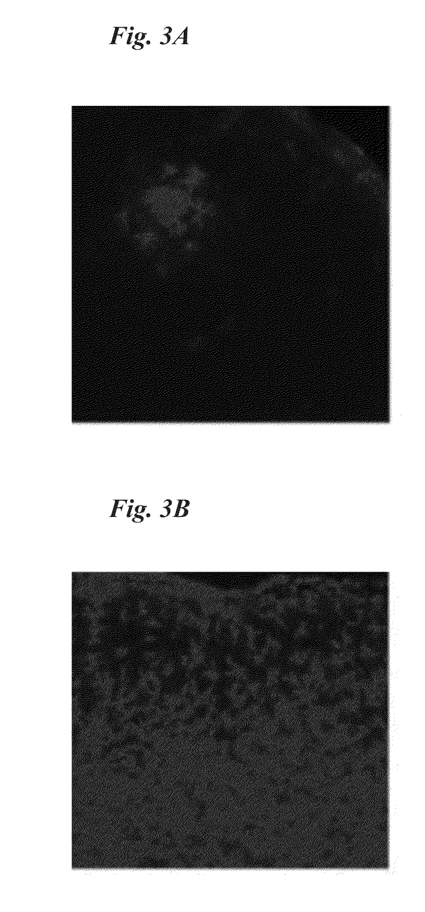

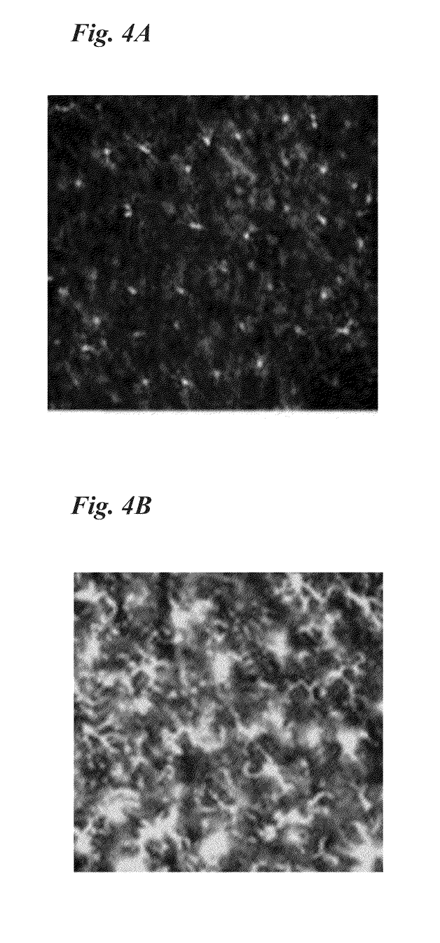

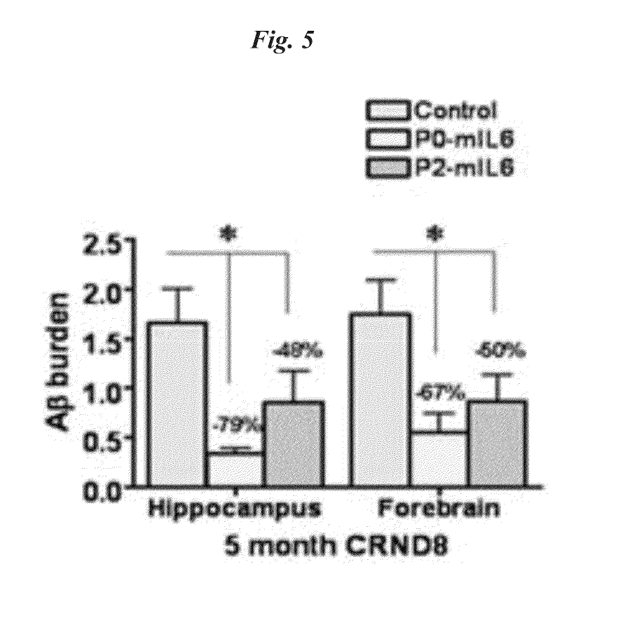

Injection of AAV1-mIL-6 into TgCRND8 mice at ages P0, P2, and 4 months resulted in increased levels of mIL-6, as indicated by sandwich ELISA (FIG. 1A-C). Astrogliosis in brains injected with AAV1-mIL-6 was increased compared to brains injected with AAV1-EGFP, as indicated by GFAP immunoreactivity (FIGS. 2A-C and FIGS. 3A-B). Microgliosis was demonstrated by increased immunoreactivity of the microglia marker Iba-1 (FIGS. 4A-B). There was a significant decrease in total forebrain Aβ as well as hippocampal Aβ plaque burdens in both P0→5 mo and P2→5 mo injection groups as compared with control mice (FIG. 5).

Chronic overexpression of mIL-6 using AAV1 induced a massive reactive gliosis in transgenic mice, suggesting that mIL-6-induced neuroinflammation does not exacerbate Aβ plaque pathology and that the observed attenuation in Aβ levels is most likely due to enhanced Aβ phagocytosis by microglia. These data refute the previous hypothesis that a strong proinflammatory stimulus like IL-6 creates a self-reinforcing neurotoxic feedback loop that promotes Aβ deposition.

### Example 2

### Microglial Phagocytosis of Aβ Aggregates In Vitro Methods

Primary mouse microglia cells were obtained from the cerebral cornices of neonate mice (1-2 days old) as described previously (Bard et al., Nat. Med., 6:916 (2000)). All studies were conducted on cultures in which >95% of cells were positive for cd11b. Hilyte 488 labeled Aβ42 (AnaSpec, Freemont, Calif., USA) was aggregated in PBS buffer at 37° C. for 6 h and then sonicated (3×10 s burst) to generate smaller fibrillar structures. Microglia pretreated with mIL-6 (R&D Systems, Minneapolis, Minn., USA) for 4 h, were treated with Hilyte 488-Aβ42 for 10 min at 37° C. Cells were washed, fixed, and mounted for visualization. For FACS analysis, after washes, glia were collected by trypsinization and resuspended in FACS buffer containing BSA. The scan was collected using Becton-Dickinson FacsVantage SE and analyzed with DIVA software (BD Biosciences).

### Results

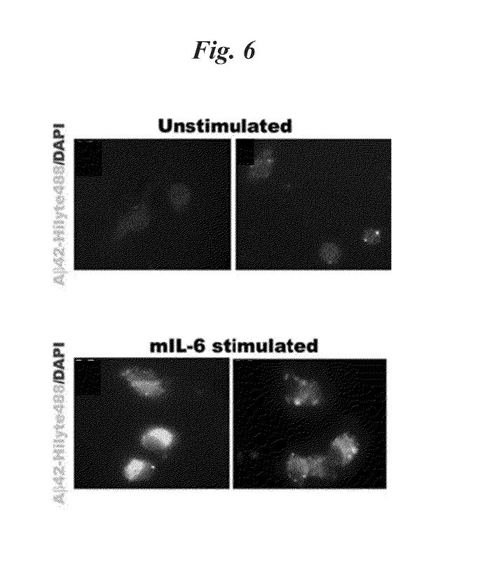

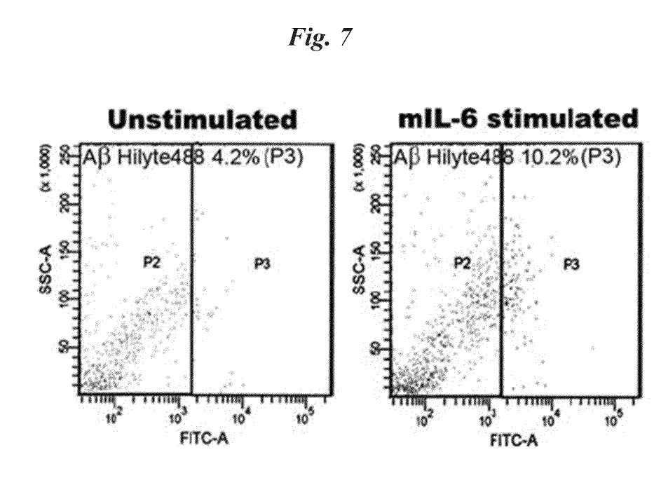

Primary wild-type mouse microglia treated with recombinant mIL-6 exhibited increased internalization of fluorescent tagged fibrillar Hilyte 488-Aβ42 compared with untreated glia (FIG. 6). FACS analysis was used to quantify the percentage of glial population positive for phagocytosed Hilyte 488 fAβ42. After fAβ42 uptake, cells were trypsinized to degrade any cell-surface-associated Hilyte 488 fAβ42. FACS analysis showed that 10.2% of mIL-6-primed glial cells were positive for fAβ42-Hilyte 488 compared with only 4.2% in untreated microglial cells (FIG. 7).

These results demonstrate an increased uptake of Aβ42 in the presence of mIL-6 and demonstrate that the reduction in Aβ deposition in mice is likely mediated by enhanced microglial clearance of the Aβ deposits.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing or suppressing amyloid deposition in central nervous system tissue of a mammal, wherein said method comprises administering a therapeutically effective amount of a viral vector capable of expressing an interleukin-6 polypeptide to said mammal.

2. The method of claim 1, wherein said viral vector is a recombinant adeno-associated virus serotype 1 viral vector.

3. The method of claim 1, wherein said interleukin-6 polypeptide is a recombinant human interleukin-6 polypeptide.

4. The method of claim 1, wherein expression of said interleukin-6 polypeptide is driven by a cytomegalovirus enhancer or a chicken β-actin promoter.

5. The method of claim 1, wherein said mammal is a human.

6. The method of claim 1, wherein said amyloid deposition is associated with Alzheimer's disease.

7. A method for reducing or suppressing amyloid deposition in central nervous system tissue of a mammal, wherein said method comprises administering a therapeutically effective amount of an interleukin-6 polypeptide or an analog thereof to said mammal.

8. The method of claim 7, wherein said interleukin-6 polypeptide is a recombinant human interleukin-6 polypeptide.

9. The method of claim 7, wherein said interleukin-6 analog is an analog derived from a recombinant human interleukin-6 polypeptide, and wherein said analog has the amyloid deposition suppression activity of a recombinant human interleukin-6 polypeptide.

10. The method of claim 7, wherein said interleukin-6 analog is a synthetic polypeptide analog of interleukin-6, and wherein said synthetic polypeptide analog has the amyloid deposition suppression activity of a recombinant human interleukin-6 polypeptide.

11. The method of claim 7, wherein said administration is a parenteral administration.

12. The method of claim 7, wherein said administration is a subcutaneous administration.

13. The method of claim 7, wherein said administration is an enteral administration.

14. The method of claim 7, wherein said interleukin-6 polypeptide comprises a modification to increase blood brain barrier permeability.

15. The method of claim 7, wherein said mammal is a human.

16. The method of claim 7, wherein said amyloid deposition is associated with Alzheimer's disease.

17. A method for reducing the level amyloid deposition in the central nervous system of a mammal or suppressing the accumulation of amyloid deposition in the central nervous system of a mammal, wherein said method comprises administering an interleukin-6 polypeptide or analog thereof to said mammal under conditions wherein said level is reduced or said accumulation is suppressed.

18. The method of claim 17, wherein said administering step comprises administering a composition containing said interleukin-6 polypeptide or said analog thereof to said mammal.

19. The method of claim 17, wherein said administering step comprises administering a composition containing a nucleic acid encoding said interleukin-6 polypeptide or said analog thereof to said mammal.

20. The method of claim 17, wherein said administering step comprises administering said interleukin-6 polypeptide or analog thereof to said mammal under conditions wherein said level is reduced.
